(19) United States
(12) Patent Application Publication
(10) Pub. No.: US 2007/0072167 A1
(43) Pub. Date: Mar. 29, 2007

(54) Tissue binding composition

(75) Inventor: Philippe Rochaix, Toulouse (FR)

Correspondence Address: FOLEY AND LARDNER LLP;SUITE 500, 3000 K STREET NW, WASHINGTON, DC, 20007, US

(73) Assignee: INSTITUT CLAUDIUS REGAUD, 131052 TOULOUSE CEDEX 3, FRANCE (FR)

(21) Appl. No.: 10/548,567

(22) PCT Filed: Mar. 12, 2004
(86) PCT No.: PCT/FR04/00608

§ 371 Date: Sep. 5, 2006

(30) Foreign Application Priority Data
Mar. 12, 2003 (FR) 0303043

(51) Int. Cl. C12Q 1/00; G01N 33/48

(52) U.S. Cl. 435/4; 435/40.5

(57) ABSTRACT

The invention relates to a tissue binding composition. The inventive composition is characterised in that it comprises: between 40 g/l and 80 g/l trehalose, preferably approximately 60 g/l; between 40% and 80% (v/v) ethanol, preferably approximately 70% (v/v): between 0% and 5% (v/v) acetic acid, preferably approximately 1% (v/v); and between 20% and 60% (v/v) water, preferably approximately 29% (v/v).

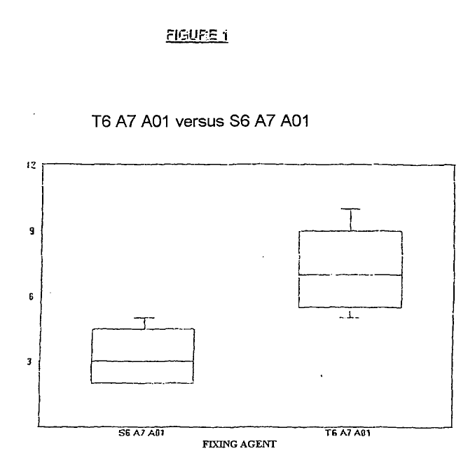

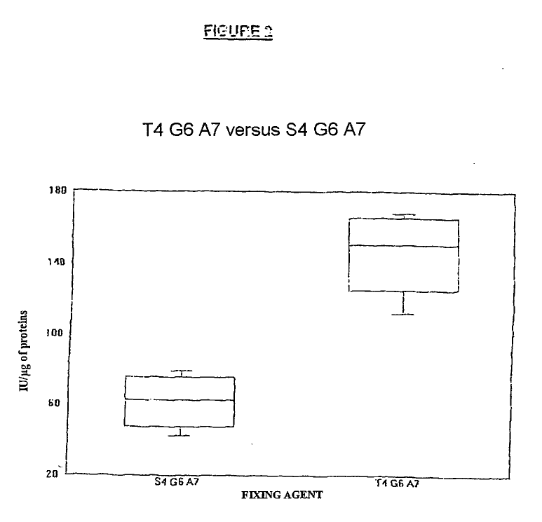

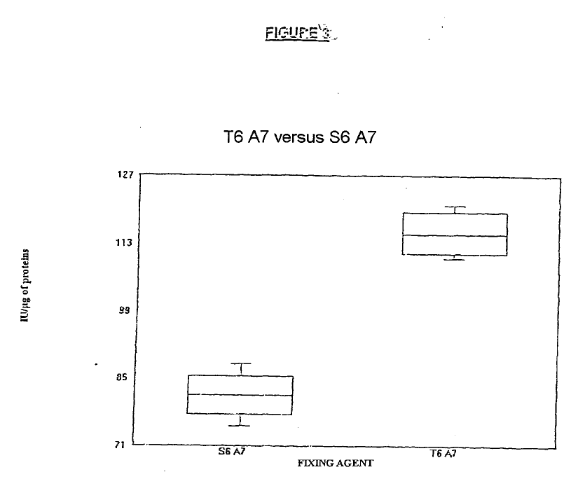

The present invention relates to the field of the preservation of tissue samples under optimal conditions, so as to be able to subsequently perform molecular biology analyses, in particular using the tissues thus preserved, for diagnostic or therapeutic purposes.

A subject of the present invention is a tissue fixing composition that makes it possible to preserve, within the tissue thus fixed, proteins and nucleic acids so as to allow their analysis in situ or their subsequent extraction from the tissue in question for analytical purposes.

In terms of tumor pathology, the quantitative and qualitative analysis of gene expression in tissue samples can provide important information relating to the physiopathological mechanisms, such as the inflammatory response, or cell growth or differentiation. Progress in the knowledge of such phenomena has resulted in advances in terms of both diagnosis and treatment. While the freezing of tissue samples (at a temperature of less than or equal to −80° C.) remains the reference technique for molecular biological analysis, its restrictive nature is recognized by everyone. This is because this technique not only requires suitable installations for storing the frozen tissues, but also imposes drastic conditions for the transport of said tissues so as not to break the cold chain.

In fact, while, for common anatomical-pathological diagnosis, the fixing and the paraffin-embedding of tissue samples are widely used since they can be readily handled, in particular for the conservation of specimens, it should be underlined that, in a diagnostic context, freezing remains however essential for certain analyses such as muscle histoenzymology or research for fusion transcripts. Some monoclonal antibodies used in immunohistochemistry for diagnostic purposes can also only be used on frozen tissue sections.

In this objective, the development of novel techniques for fixing and processing tissue samples that allow a complete preservation not only of the proteins, but also of the main target for the molecular biological analysis of the tissue samples—namely the nucleic acids (DNA or RNA)—could remedy the drawbacks of the prior art.

The advent of a novel fixing technique that preserves the proteins and nucleic acids of paraffin-embedded tissues would therefore offer many applications, such as, for example, the analysis of cell populations defined by microdissection (Fend 1999).

As the technique currently stands, three families of products are used for the fixing of biological tissues:

aldehydes (formol, paraformaldehyde, glutaraldehyde, etc.), which form covalent bonds with biological molecules, stabilizing them and inhibiting enzymatic activities,

alcohols (ethanol, methanol), which dehydrate the tissues, and

acids, in particular acetic acid, which decrease shrinking phenomena due to the alcohols and which precipitate the proteins.

Many fixing mixtures can be used: formol alone, AFA (alcohol+formol+acetic acid), Bouin's liquid (formol+picric acid+acetic acid), Duboscq-Brazil liquid (alcohol+formol+picric acid), etc.

As indicated above, it is important to be able to conserve, with or without storage, tissue samples, especially animal and in particular human tissue samples, in such a way as to preserve, in a state as close as possible to their natural state, the elements of said tissues for which the analysis will subsequently be carried out.

Such analyses will be carried out on proteins and/or nucleic acids extracted from the fixed and conserved tissues, for diagnostic or therapeutic purposes, or even for the purposes of studies of certain tissues such as tumors. These analyses therefore require that the elements to be analyzed be extracted with suitable yields and in the absence of any contaminants.

The problem is however a little different depending on whether the intention is to extract, from the fixed and conserved tissues for analytical purposes, nucleic acids or proteins.

Three essential conditions are required for the analysis of RNA on fixed and paraffin-embedded tissues: 1) a good RNA extraction yield, 2) a good quality of the RNA extracted, and 3) the absence of contamination with genomic DNA (Shibutani 2000). To date, the extraction and analysis of total RNA from formol-fixed and paraffin-embedded tissues have essentially been applied for the detection of viral RNAs, in the context of hepatitis C for example (Dries 1999; Guerrero 1997).

However, RNA degradation and insufficient extraction considerably impair the analysis, in particular quantitative analysis, of formol-fixed and paraffin-embedded tissue samples (Rupp 1988; Stanta 1991, Finke 1993). In addition, contamination with genomic DNA is a commonly encountered problem (Foss 1994); the secondary use of DNase treatments after phenol/chloroform extraction and ethanol precipitation of extracted RNA samples can considerably reduce the amount of final RNA.

In general, several RT-PCR feasibility studies have shown that nonbridging fixing agents such as acetone or Methacarn (methanol, chloroform, acetic acid) are superior in terms of product amplification efficiency, compared with formaldehyde fixing agents (Koopmans 1993; Tyrrell 1995). Fixing with acetone (Sato 1991; Sato 1992) gives excellent RNA extraction yields but, besides the restrictive nature of this technique (fixing at −80° C.), the level of genomic DNA contamination can be high (Shibutani 2000).

It is within this context that novel fixing agents based on protein precipitation have emerged, such as methacarn (Puchtler 1970; Mitchell 1985; Shibutani 2000), which agents preserve the RNA and the proteins intact, but which are not devoid of a certain toxicity that limits their common use. As regards paraffin-embedding, one of the main stumbling blocks encountered is the obtaining of RNA of large size. Moreover, the contamination of extracts with genomic DNA is reported to a greater extent for fixed and paraffin-embedded tissues than for frozen tissues (Rupp 1988; Ben-Ezra 1991; Von Wiezsäcker 1991; Foss 1994). However, it would appear that this drawback is prevented with the novel fixing agents (methacarn, for example) (Shibutani 2000).

Unlike the analysis of nucleic acids, very few studies have related to the possibility of extracting the proteins from fixed and paraffin-embedded tissues (Hara 1993; Ikeda 1998). This limited knowledge is probably explained by the fact that formaldehyde fixing agents, which are the most widely used, create interprotein bonds. Here again, the nonbridging fixing agents (acetone, ethanol, methacarn) open up new perspectives (Rognum 1980; Orstavik 1981; Mitchell 1985; Conti 1988). Acetone-type fixing agents do not affect the quality of proteins, but the methodological constraints are considerable without offering any major advantage compared with conventional freezing.

Methacarn has shown itself to be effective (Shibutani 2000), but the main disadvantage of this novel fixing agent is the considerable toxicity of its constituents. Moreover, the paraffin-embedding and deparaffinizing steps do not appear to have a major influence on the quality of the protein extraction when the tissues have been fixed using nonbridging fixing agents (Shibutani 2000).

The present invention proposes to remedy the drawbacks of the prior art by proposing in particular a novel tissue fixing composition based on nontoxic chemical compounds that preserve cell and tissue structures. In addition, the tissue fixing composition of the invention provides easy handling since it allows tissue samples to be conserved in a paraffin block or in dehydrated form in particular.

The particularity of the tissue fixing composition of the invention is based on the fact that it comprises trehalose combined with other compounds. More particularly, this composition comprises:
trehalose at a concentration between 40 and 80 g/l, preferably between 40 and 70 g/l, and even more preferably approximately 60 g/l,
ethanol in an amount of between 40% and 80% (v/v), preferably between 55% and 75% (v/v), and even more preferably approximately 70% (v/v),
acetic acid in an amount of between 0% and 5% (v/v), preferably between 0.5% and 3% (v/v), and even more preferably approximately 1% (v/v), and
water in an amount of between 20% and 60% (v/v), preferably between 25% and 50% (v/v), and even more preferably approximately 29% (v/v).

The inventors have, moreover, demonstrated that the preparation of the abovementioned tissue fixing composition can be carried out optimally in two steps. This involves, first of all, preparing a mixture with only 45% of ethanol (stable at 0° C.) and adding pure ethanol (also stable at 0° C.) at the time the final composition is prepared, in a sufficient amount to obtain the tissue fixing composition of the invention as specified above. This makes it possible in particular to stabilize the products of preparation of said composition and to store them in a cold environment in order therefore to use them already cooled from the beginning of fixing.

By way of example, to prepare a liter of the tissue fixing composition of the invention, it is possible to prepare, in a first step, a mixture comprising 60 g of trehalose, 10 ml of acetic acid, 300 ml of water and 250 ml of ethanol, it being understood that it will then be necessary to add thereto 450 ml of pure ethanol.

In a second step, the abovementioned mixture and the pure ethanol must be mixed just before use of the final composition, in a proportion of 5.5 parts of the mixture and 4.5 parts of ethanol. Thus, the final tissue fixing composition comprises: 60 g of trehalose, 10 ml of acetic acid, 300 ml of water and 700 ml (250+450) of ethanol, which correctly corresponds to the composition indicated above.

As demonstrated hereinafter, by means of the comparative analyses carried out in the context of various experiments using, firstly, the fixing composition of the invention and, secondly, the usual fixing agent, namely AFA, it is clear that the results obtained are much better when they are realized on tissues having been fixed beforehand with the fixing composition of the invention. This is apparent in particular in table 1, showing an increased sensitivity of the detection of antigenic sites using the tissues fixed with the fixing composition of the invention, but also in the extraction yields obtained from such tissues.

According to a particular embodiment of the present invention, the abovementioned tissue fixing composition also comprises glycerol in an amount of between 0% and 10% (v/v), preferably between 3% and 8% (v/v), and even more preferably approximately 6%.

By way of example, the composition of the invention may in this case comprise 40 g/l of trehalose, 70% of ethanol (v/v), 6% of glycerol (v/v), 1% of acetic acid (v/v) and 23% of water (v/v).

The glycerol is in fact only added to the tissue fixing composition of the invention when a greater concentration of alcohol is necessary, in particular with the aim of conserving nucleic acids of very good quality.

The tissue fixing composition of the invention comprising glycerol makes it possible in particular, through the osmotic action of the glycerol, to reduce the amount of trehalose used and to prevent precipitation thereof in a medium with a higher alcohol concentration.

A subject of the present invention is also a tissue fixing method consisting in immersing the fresh tissue samples in the tissue fixing composition of the invention, at a temperature of between 0 and 6° C., preferably between 0 and 4° C., and even more preferably at a temperature of approximately 1° C., for at least 12 hours, preferably at least 16 hours, and even more preferably for approximately 20 hours, depending on the thickness of the tissue.

The term “fresh tissue sample” is intended to mean any tissue sample resulting in particular from an animal, including human, specimen taken under standard conditions well known to those skilled in the art, it being understood that the fixing should take place as rapidly as possible after tissue devascularization in order to obtain the best preservation of the quality of the nucleic acids, and in particular of the RNA.

In fact, not only have the inventors demonstrated that the use of the tissue fixing composition of the invention makes it possible to perform a finer analysis that gives better results and/or yields than those obtained using a fixing agent of the prior art, but they have further improved the method of processing a tissue sample, with the aim of further improving the quality of the subsequent biological analyses.

Thus, the inventors have demonstrated that a tissue sample fixed for the purposes of subsequent analyses can be prepared in the context of the present invention according to a method of processing comprising the steps consisting in:
a) fixing the sample by means of the tissue fixing composition of the invention,
b) dehydrating the sample obtained in a),
c) conserving the sample obtained in b),
   i) in the dehydrated state,
   ii) in a resin, or
   iii) by paraffin-embedding.

Entirely surprisingly, the inventors have demonstrated that the conservation of said sample in dehydrated form after fixing thereof by means of the composition of the invention makes it possible not only to perform in situ analyses of a quality of at least equivalent to that of the other known techniques, but also makes it possible to perform nucleic acid extractions of much better quality and more readily than by using said techniques of the prior art. It is in fact the combination of the use of the tissue fixing composition of the invention and of the conservation of the sample thus fixed in the dehydrated state that has made it possible to obtain such results.

In fact, the dehydration and the maintaining in this state of a tissue sample fixed beforehand with the fixing composition of the invention comprising in particular trehalose makes it possible to preserve not only the cell and tissue morphology of the sample, but also the cellular functions such as enzymatic functions. The inventors have, moreover, demonstrated, as indicated hereinafter, that RNAs extracted from tissue samples fixed with the composition of the invention and conserved in the dehydrated state are of better quality than when said samples, even fixed with the tissue fixing composition of the invention, have been paraffin-embedded.

Moreover, the inventors have also demonstrated that the “conventional” methods of paraffin-embedding samples can also be improved in the context of the present invention. Thus, a tissue sample can be prepared in accordance with a method of processing comprising the steps consisting in:
a) fixing the sample by means of the tissue fixing composition of the invention,
b) dehydrating the sample obtained in a),
c) soaking the sample obtained in b), in paraffin,
d) paraffin-embedding the sample obtained in c),
e) obtaining “sections” from the embedding block obtained in d) (by cutting the embedding block into extremely thin “slices” a few microns thick),
f) spreading a section obtained in e) out on a glass slide and making this section adhere,
g) deparaffinizing the section thus made to adhere,
h) staining said section.

The biological analyses are then carried out using this section, both in terms of the visualization of certain constituent elements of the tissue thus prepared and in terms of the extraction of the nucleic acids or proteins.

At the end of step f) above, i.e. after a section obtained after cutting the paraffin-embedding block has been spread out and made to adhere, the slide is referred to as a “white slide” since it is not stained.

Conventionally, the deparaffinization of the section is carried out using successive xylene baths. The tissue sample is then gradually rehydrated by means of successive baths of alcohol at 100° C., 80° C. then 50° C., and, finally, in water. After this rehydration step, the tissue sample is stained.

The inventors have determined, surprisingly, that it is possible to obtain even better results in terms of preservation of the morphology of the tissue sample if the white slide, before the deparaffinizing step, is immersed in a bath comprising 75% (v/v) of alcohol, 2% (v/v) of formol, 5% (v/v) of acetic acid, 1% (v/v) of Tween 20® and 17% (v/v) of water, for approximately 5 minutes at ambient temperature.

This is because such an additional treatment makes it possible to preserve good cell morphology, that is particularly useful in certain cases, such as the analysis of lymphoid subpopulations, for example.

The present invention relates to any type of tissue sample to be fixed and/or processed, in particular animal, including human, tissue samples that may be pathological or nonpathological. The invention applies, for example, to the fixing and/or processing of tumor tissues, in particular in the context of the forming of a tumor library.

The results of experiments hereinafter will make it possible to understand the invention more clearly. They are however only mentioned purely by way of illustration.

### EXAMPLE 1

### Paraffin-Embedded Samples

1. Tissue Processing:

a) Freezing:

Fresh tissue samples are placed in cryotubes and immersed directly in liquid nitrogen (−196° C.) and are then conserved in a freezer at −80° C.

b) Fixing and Embedding:

Fresh tissue samples are immersed in the tissue fixing composition of the invention at approximately +1° C. for at least 12 hours. They are then dehydrated in 3 or 4 successive baths of absolute alcohol for one hour each at a temperature of between 0 and 4° C., and then in a bath of acetone for approximately 1 h at approximately 4° C. and in 2 baths of acetone for approximately 1 h each at ambient temperature.

The dehydrated samples are incubated in liquid paraffin at 58° C. for 10 to 15 hours, preferably 12 hours and embedded in cassettes (standard anatomical-pathological technique).

c) Deparaffinizing:

The samples are sectioned as strips on the microtome and are then deparaffinized using 2 successive xylene baths and 2 absolute ethanol baths.

2. Protein Analysis

a) Extraction and Assaying:

First Extraction Method:

The extraction uses a lysis buffer (100 mM Tris HCl, pH 7.4, 2% SDS, protease inhibitor™ Sigma) at 95° C. for 5 minutes and then sonification.

Second Extraction Method:

The extraction uses a lysis buffer (50 mM Tris HCl, pH 7.5, 150 mM NaCl, 1% Nonidet P40, protease inhibitor™ Sigma) at 4° C. and grinding.

Since the protein assay cannot be carried out according to the Bradford technique because of the large amounts of SDS, an assay procedure based on bicinchoninic acid and copper sulfate was used.

b) Analysis:

The protein samples are resolved on a 10% polyacrylamide gel and transferred onto a PVDF membrane.

If only the overall protein profile is sought, the membrane is stained by incubation for one hour in a solution of Amidoblack™ (0.1% Amidoblack™, Sigma, 45% ethanol, 10% acetic acid).

If an immunodetection is desired, the membrane is incubated with a primary antibody and then a secondary antibody, and is then visualized by chemiluminescence (ECL+™, Pierce) according to the manufacturer's recommendations.

3. DNA Extraction

This is a conventional phenol/chloroform extraction after deparaffinizing or sectioning of the frozen samples.

4. RNA Extraction

This is a conventional Trizol™ extraction after deparaffinizing or sectioning of the frozen samples.

Results

1. Morphology

5 μm thick tissue sections were cut, spread on slides, deparaffinized, and then stained with hemalin-eosin for a morphological study. The inventors obtained a histological and cytological morphology of very good quality, with respect to both the epithelial and connective constituents, comparable to that observed in common techniques. In order to increase the reproducibility of the quality of the stainings obtained, a post-fixation step (optional) can be carried out: after the sections have been spread on the slides, the slides are immersed, before deparaffinizing, for 5 minutes in AFA to which 1% of Tween 20 has been added, and then rinsed with water.

2. Immunohistochemical Study

The quality of antigenic site preservation was evaluated by immunohistochemical study using a large battery of monoclonal antibodies (table 1). This study was carried out on paraffin sections, spread on slides and deparaffinized, without reactivation of antigenic sites (no microwaving or enzymatic digestion), even though this reactivation was recommended by the antibody supplier. The peroxidase activity visualization was carried out using the LSAB kit (Dako). For each antibody, the labeling obtained on the same tissue type was analyzed comparatively, between the usual fixing agent (AFA) and the fixing composition of the invention (table 1).

TABLE 1
Comparative immunohistochemical study

| | | | Labeling | Labeling |
|---|---|---|---|---|
| | | | intensity | intensity |
| | | | Fixing | Usual |
| | | | composition | fixing |
| | | Recommended | of the | agent |
| Antibody | Dilution | pretreatment | invention | (AFA) |
| ACE^{#} | 1/6 | No | +++ | + |
| (Dako) | | | | |
| Collagen | 1/50 | Proteinase K | +++ | + |
| IV^{#} | | | | |
| (Dako) | | | | |
| CK5/6^{#} | | No | +++ | 0 |
| (Dako) | | | | |
| CK19^{#} | 1/100 | No | +++ | + |
| (Dako) | | | | |
| Bc12^{§} | 1/50 | Microwaves | +++ | + |
| (Dako) | | | | |
| Calcitonin^{§} | 1/10 | No | +++ | + |
| (Dako) | | | numerous C | rare C |
| | | | cells | cells |
| TTF-1^{§} | 1/50 | Microwaves | +++ | 0 |
| (Microm) | | | | |
| CD3^{£} | 1/200 | Microwaves | +++ | + |
| (Dako) | | | | |
| CD15^{£} | 1/50 | No | +++ | ++ |
| (immunotech) | | | | |
| CD30^{£} | 1/2 | Microwaves | +++ | ++ |
| (Dako) | | | numerous | a few |
| | | | cells | cells |
| CK20* | 1/100 | Microwaves | +++ | +++ |
| (Dako) | | | 100% cells | 50% cells |
| KI-67^{$} | 1/50 | Microwaves | +++ | 0 |
| (Dako) | | | | |
| PCNA^{$} | | No | ++ | 0 |
| (Dako) | | | | |
| P53^{$} | 1/50 | Microwaves | ++ | 0 |
| (Dako) | | | | |

^{#}reactivity studied on breast,
^{§}reactivity studied on thyroid,
^{£}reactivity studied on a lymph node,
*reactivity studied on colon,
^{$}reactivity studied on a sarcoma,
+++ = intense labeling,
++ = moderate labeling,
+ = weak labeling,
0 = absence of labeling.

Thus, it appears that the immunoreactivity observed with the tissue fixing composition of the invention is greater than that observed with the usual fixing agent (AFA) for all the antibodies tested. For the immunohistochemical study, the use of the composition of the invention makes it possible to eliminate the pretreatment, intended to unmask the antigenic sites, even though said pretreatment is recommended by the supplying laboratory. In addition, if the immunoreactivity obtained with the composition of the invention without pretreatment is compared with that obtained with the usual fixing agent according to the usual recommendations (microwaves), a more intense labeling is also observed with the use of the fixing composition of the invention.

3. Protein Analysis

The extraction yields differ according to the type of extraction buffer and the fixing agent used.

TABLE 2
Protein extraction of a human uterine leiomyoma

| | “NP40” buffer | | “SDS” buffer | |
|---|---|---|---|---|
| Freezing | 200 | μg prot/mg of | 310 | μg prot/mg of |
| | | tissue | | tissue |
| Fixing | 125 | μg prot/mg of | 207 | μg prot/mg of |
| composition of | | tissue | | tissue |
| the invention | | | | |
| AFA | 2.5 | μg prot/mg of | 5.77 | μg prot/mg of |
| | | tissue | | tissue |

The composition of the invention always maintains yields that are less than those of freezing, but gives better results than AFA.

The protein profiles obtained from tissues fixed with the composition of the invention also differ according to the extraction buffer used: many bands are missing on the gels produced from the extractions with NP40 buffer, whereas those produced from extraction with SDS buffer are relatively similar to those from freezing (data not shown).

The proteins obtained from the extraction with the SDS buffer were recognized, at the expected size, in immunoblotting, by antibodies specific for cytosolic (actin and desmin), nuclear (estrogen receptor=RE) and mitochondrial (Bcl2) proteins. While the intensity of the labeling is identical for the freezing and the composition of the invention, it decreases for the high molecular weight proteins such as RE for AFA (data not shown).

DNA

DNA extraction tests were carried out on tissue samples that had been fixed according to the technique of the invention, fixed in AFA, or frozen, a few days after fixing of the samples and were then repeated a year later.

The amount of DNA extracted from the tissues fixed with the composition of the invention were similar to that of the frozen tissues, and much higher than that of the AFA-fixed tissues (table 3).

TABLE 3
DNA extraction yield on several types of
tissues fixed with the composition of the invention

| Tissue | Yield | |
|---|---|---|
| Ovary | 7.46 | μg DNA/mg of tissue |
| Melanoma | 18.41 | μg DNA/mg of tissue |
| Lymphoma | 6 | μg DNA/mg of tissue |

The DNAs extracted during these manipulations were very high molecular weight DNAs. The inventors were able to PCR-amplify a sequence of 2800 bp of the BRCA-1 gene. The tissue fixing technique of the invention is therefore compatible with the extraction and analysis of DNA from tissues thus fixed and paraffin-embedded. Migration of the total DNA on a 0.8% agarose gel confirms the large size of the DNA fragments obtained (data not shown).

RNA

RNA extraction tests were carried out on tissue samples that had been fixed according to the technique of the invention, fixed in AFA or frozen, a few days after the fixing of the samples and were then repeated a year later.

The amount of RNA extracted from the tissues fixed with the composition of the invention is similar to that of the frozen tissues, and much higher than that of the AFA-fixed tissues (table 4).

TABLE 4
Mouse liver RNA extraction yield

| Fixing | | |
|---|---|---|
| agent | Yield | |
| Freezing | 1.3 ± 0.4 | μg RNA/mg of |
| | | tissue |
| Fixing | 1.1 ± 0.2 | μg RNA/mg of |
| composition | | tissue |
| of the | | |
| invention | | |
| AFA | 0.017 ± 0.10 | μg RNA/mg of |
| | | tissue |

The bands corresponding to the 28S and 18S ribosomal RNAs are visible, attesting to good preservation of the RNAs. The use of the fixing composition of the invention made it possible to obtain the same RNA profile as that obtained with freezing, before paraffin-embedding.

The quality of the RNA obtained and the absence of contaminating DNA were evaluated by RT-PCR-amplification of the Raf gene.

### EXAMPLE 2

### Comparative Extraction of RNA

Tissue samples were fixed by means of the tissue fixing composition of the invention, and then dehydrated.

These samples were then divided up into two equivalent groups, one of the groups was subjected to paraffin-embedding, the other was subjected to conservation in dehydrated form in a hermetic container containing a desiccating agent.

The RNAs were then extracted from these various samples, resolved on a 0.8% agarose gel, and compared with those extracted from frozen samples.

It is found that the RNAs extracted from the paraffin blocks show a lightening of the bands corresponding to the ribosomal RNAs and the appearance of a slight smear evoking a partial degradation and/or a contamination with DNA. Identical results are obtained after the samples have been conserved for a year. However, the RNAs extracted from the samples conserved in dehydrated form exhibit a better quality in the sense that they in particular appear more clearly on the gel (data not shown).

Thus, it appears that the paraffin-embedding of the samples is accompanied by a partial degradation of the messenger RNAs and by a possible contamination with genomic DNA. Therefore, when the study of nucleic acids is of utmost importance, it appears to be preferable to conserve the fixed samples in dehydrated form in an anhydrous medium, without paraffin-embedding them.

### EXAMPLE 3

### Demonstration of the Specific Protective Effect of Trehalose During the Dehydration of Proteins, Comparison with Sucrose

In order to evaluate the functional protection of proteins conferred by trehalose, the activity of a liver enzyme (TGO) was measured and compared in protein extracts obtained from tissues fixed with various tissue fixing compositions.

The formula of the tissue fixing composition of the invention comprises rehalose, ethanol, acetic acid and water (called T6 A7 A01) and can be provided in two forms, one comprising glycerol but not acetic acid (called T4 G6 A7) and the other devoid of acetic acid (called T6 A7).

Six fixing compositions were prepared as indicated hereinafter:
three fixing compositions of the invention comprising trehalose:
   1. T6 A7 A01: 60 g/l trehalose; 1% (V/V) acetic acid; 70% (V/V) ethanol; 29% (V/V) water,
   2. T4 G6 A7: 40 g/l trehalose; 6% (V/V) glycerol; 70% (V/V) ethanol; 24% (V/V) water,
   3. T6 A7: 60 g/l trehalose; 70% (V/V) ethanol; 30% (V/V) water.
three other fixing compositions with the same formulation but in which the trehalose has been replaced with another disaccharide: sucrose:
   4. S6 A7 A01: 60 g/l sucrose; 1% (V/V) acetic acid; 70% (V/V) ethanol; 29% (V/V) water,
   5. S4 G6 A7: 40 g/l sucrose; 6% (V/V) glycerol; 70% (V/V) ethanol; 24% (V/V) water,
   6. S6 A7: 60 g/l sucrose; 70% (V/V) ethanol; 30% (V/V) water.

A fresh liver from a nude mouse was dissected into six equivalent fragments, and each of them was fixed in one of the abovementioned six fixing compositions, according to the protocol given in the above description. The samples were dehydrated and stored at 4° C.

This protocol was carried out four times, on different days, using livers from different mice.

The proteins were extracted using a weakly denaturing extraction buffer (50 mM Tris HCl, pH 7.5, 150 mM NaCl, 1% Nonidet P40, protease inhibitor™ Sigma) at 4° C. and assayed according to the Bradford technique.

The TGO enzymatic activity was measured using a Konelab 5.0.5 automatic device. The results were weighted by the amount of protein and given in the form of international units/microgram of protein (IU/μg of proteins).

These results were compared in pairs (three binomes) according to the presence of trehalose or sucrose in the tissue fixing composition tested, everything otherwise being equal:
T6 A7 A01 versus S6 A7 A01,
T4 G6 A7 versus S4 G6 A7, and
T6 A7 versus S6 A7.

The results were compared according to the Dunnett method for comparing means after analysis of variance.

The results obtained are given in the tables below and are illustrated by the corresponding figures.

[image]

[image]

[image]

### CONCLUSION

Whatever the tissue fixing composition tested, the presence of trehalose makes it possible to obtain much better results with respect to preservation of the enzymatic activity of mouse liver TGOs, something which sucrose does not do.

### REFERENCES

Ben-Ezra J, Johnson D A, Rossi J, Cook N, Wu A. Effect of fixation on the amplification of nucleic acids from paraffin-embedded material by the polymerase chain reaction. J Histochem Cytochem 1991; 39 : 351-4.
Conti C J, Larcher F, Chesner J, Aldaz C M. Polyacrylamide gel electrophoresis and immunoblotting of proteins extracted from paraffin-embedded tissue sections. J Histochem Cytochem 1988; 36 : 547-50.
Dries V, Von Both I, Muller M, Gerken G, Shirmacher P, Odenthal M, Bartenschlager R, Drebber U, Meyer Zum Buschenfeld K H, Dienes H P. Detection of hepatitis C virus in paraffin-embedded liver biopsies of patients negative for viral RNA in serum. Hepatology 1999; 29 : 223-9.
Fend F, Emmert Buck M R, Chuaqui R, Cole K, Lee J, Liotta L A, Raffeld M. Immuno-LCM: laser capture microdissection of immunostained frozen sections for mRNA analysis. Am J Pathol 1999; 154 : 61-6.
Fink J, Fritzen R, Ternes P, Lange W, Dölken G. An improved strategy and a useful housekeeping gene for RNA analysis from formalian-fixed, paraffin-embedded tissues by PCR Bio Techniques 1993; 14 : 448-53.
Foss R D, Guha Thakurta N, Conran R M, Gutman P. Effects of fixative and fixation time on the extraction and polymerase chain reaction amplification of RNA from paraffin-embedded tissue. Comparison of two housekeeping gene mRNA controls. Diagn Mol Pathol 1994; 3 : 148-55.
Guerrero R B, Batts K P, Brandhagen DJ, Germer J J, Perez R G, Persing D H. Effects of formalin fixation and prolonged block storage on detection of hepatitis C virus RNA in liver tissue. Diagn Mol Pathol 1997; 6 : 277-81.
Hara A, Sakai N, Yamada H, Yoshimi N, Tanaka T, Mori H. Immunoblot analysis of the placental form of glutathione S-transferase in protein extracted from paraffin-embedded human glioma Tissue. J Cancer Res Clin Oncol 1993 ; 119 : 493-6.
Ikeda K, Monden T, Kanoh T, Tsujie M, Izawa H, Haba A, Ohnishi T, Sekimoto M, Tomita N, Shiozaki H, Monden N. Extraction and analysis of diagnostically useful proteins from formalin-fixed, paraffin-embedded tissue sections. J Histochem Cytochem 1998; 46 : 397-403.
Koopmans M, Monroe S S, Coffield L M, Zaki S R. Optimization of extraction and PCR amplification of RNA extracts from paraffin-embedded tissue in different fixatives. J Virol Methods 1993; 43 : 189-204.
Mitchell D, Ibrahim S, Gusterson B A. Improved immunohistochemical localization of tissue antigens using modified methacarn fixation. J Histochem Cytochem 1985; 33 : 428-32.
Orstavik T B, Brandtzaeg P, Nustad K, Pierce J V. Effects of different tissue processing methods on the immunohistochemical localization of kallikrein in the pancreas. J Histochem Cytochem 1981; 29 : 985-8.
Puchtler H, Waldrop F S, Meloan S N, Terry M S, Conner H M. Methacarn (methanol-Carnoy) fixation. Practical and theoretical considerations. Histochemie 1970; 21 : 97-116.
Rognum T O, Brandtzaeg P, Ørjasaeter H, Fausa O. Immunohistochemistry of epithelial cell markers in normal and pathological colon mucosa. Comparison of results based on routine formalin- and cold ethanol-fixation methods. Histochemistry 1980; 67 : 7-21.
Rupp G M, Locker J. Purification and analysis of RNA from paraffin-embedded tissues. Bio Techniques 1988; 6 : 56-60.
Sato Y, Mukai K, Furuya S, Kameya T, Hirohashi S. The AmeX method: a multipurpose tissue-processing and paraffin-embedded method. Extraction of protein and application to immunoblotting. Am J Pathol 1992; 140 : 775-9.
Sato Y, Mukai K, Furuya S, Shimosato Y. AmeX method: a multipurpose tissue-processing and paraffin-embedded method. III. Extraction and purification of RNA and application to slot-blot hybridisation analysis. J Pathol 1991; 163 : 81-5.
Shibutani M, Uneyama C, Miyazaki K, Toyoda K, Hirose M. Methacam fixation: a novel tool for analysis of gene expressions in paraffin-embedded tissue specimens. Laboratory Investigation 2000; 80 : 199-208.
Stanta G, Schneider C. RNA extracted from paraffin-embedded human tissues is amenable to analysis by PCR amplification. Bio Techniques 1991; 11 : 304-8.
Tyrrell L, Elias S, Longley J. Detection of specific mRNAs in routinely processed dermatopathology specimens. Am J Dermatopathol 1995; 17 : 476-83.
Von Weizsäcker F. Labeit S, Koch H K, Oehlert W, Gerok W, Blum HE. A simple and rapid method for the detection of RNA in formalin-fixed, paraffin-embedded tissues by PCR amplification. Biochem Biophys Res Commun 1991; 174 : 176-80.

1.-7. (canceled)

8. A tissue fixing composition, comprising: trehalose at a concentration between 40 g/l and 80 g/l, ethanol in an amount of between 40% and 80% (v/v), acetic acid in an amount of between 0% and 5% (v/v), and water in an amount of between 20% and 60% (v/v).

9. The tissue fixing composition of claim 8, comprising: trehalose at a concentration of approximately 60 g/l, ethanol in an amount of approximately 70% (v/v), acetic acid in an amount of approximately 1% (v/v), and water in an amount of approximately 29% (v/v).

10. The tissue fixing composition of claim 8, further comprising glycerol in an amount of between 0% and 10% (v/v).

11. The tissue fixing composition of claim 10, wherein glycerol is present in an amount of approximately 6% (v/v).

12. A method of fixing tissue, comprising immersing at least one fresh tissue sample in the tissue fixing composition of claim 8 at a temperature of between 0 and 6° C. for at least 12 hours, whereby the tissue sample is fixed.

13. The method of claim 12, wherein the tissue sample is immersed in the tissue fixing composition at a temperature of approximately 1° C. for approximately 20 hours.

14. A method of processing a tissue sample, comprising:
a. fixing said sample with the tissue fixing composition of claim 8;
b. dehydrating the fixed sample; and
c. conserving the dehydrated sample,
wherein the dehydrated sample is conserved in the dehydrated state, in a resin, or by paraffin-embedding.

15. The method of claim 14, wherein the sample is conserved by paraffin-embedding, comprising:
c1. soaking the dehydrated sample in paraffin;
c2. paraffin-embedding the soaked sample;
c3. obtaining at least one section from the embedding block;
c4. spreading the section out on a glass slide;
c5. adhering the section to the glass slide;
c6. deparaffinizing the adhered section; and
c7. staining the deparaffinized section.

16. The method of claim 15, wherein prior to the deparaffinizing step, the adhered section is immersed in a composition comprising 75% (v/v) of alcohol, 2% (v/v) of formol, 5% (v/v) of acetic acid, 1% (v/v) of polysorbate 20, and 17% (v/v) of water.

17. The method of claim 14, wherein the sample is fixed at a temperature of between 0 and 6° C. for at least 12 hours.
